(19) United States
(12) Patent Application Publication
(10) Pub. No.: US 2023/0399699 A1
(43) Pub. Date: Dec. 14, 2023

(54) STRATIFICATION METHODS FOR ASSESSING THE PROGRESSION AND RISK OF ADVANCED COLORECTAL ADENOMA AND COLORECTAL CANCER

(71) Applicant: SOCIETE DE COMMERCIALISATION DES PRODUITS DE LA RECHERCHE APPLIQUEE SOCPRA SANTE ET HUMAINES, S.E.C., Sherbrooke (CA)

(72) Inventors: Jean-francois BEAULIEU, Sherbrooke (CA); Elizabeth Herring, North Hatley (CA); Eric Tremblay, Windsor (CA)

(21) Appl. No.: 18/034,571

(22) PCT Filed: Nov. 2, 2021
(86) PCT No.: PCT/CA2021/051548

§ 371 Date: Apr. 28, 2023

Related U.S. Application Data
(60) Provisional application No. 63/108,510, filed on Nov. 2, 2020.

(51) Int. Cl. C12Q 1/6886; G01N 33/574

(52) U.S. Cl. CPC C12Q 1/6886; G01N 33/57419; C12Q 2600/118; C12Q 2600/158; C12Q 2600/154; C12Q 2600/156

(57) ABSTRACT

The present disclosure concerns a method for stratifying the risk of a subject of having an advanced colorectal adenoma or a colorectal cancer based on determining the presence of overexpressed mRNA transcripts in the subject's stool. The method can be used to screen for subjects suitable for a colonoscopy. The method can also be used to tailor the stratified subject's treatment regimen.

### CROSS-REFERENCE TO RELATED APPLICATIONS AND DOCUMENTS

This application claims priority from U.S. provisional application 63/108,510 filed on Nov. 2, 2020 and herewith incorporated in its entirety.

### TECHNOLOGICAL FIELD

The present disclosure relates to non-invasive methods for assessing the risk of a subject oh having an advanced adenoma or a colorectal cancer based on the determination of modulation of the mRNA levels of one or more genes present in the subject's stool sample.

### BACKGROUND

Colorectal cancer (CRC) is one of the few cancer types for which screening has been proven to reduce cancer mortality in average-risk individuals. Indeed, the spread of the disease in terms of local invasion as well as to lymph nodes and distant organs at the time of the diagnosis is an important prognostic factor, with five-year survival rates of more than 90% for individuals with localized lesions but only ˜10% for those having their CRC metastasized to distal organs. Early detection is thus a key factor in reducing mortality from CRC. Advanced adenomas (AA) are also important to detect since they are considered to be the precursors of CRC while non-advanced adenomas (<1 cm without advanced histology) may not be associated with increased colorectal cancer risk. Several screening regiments for CRC and AA are recommended such as fecal occult blood testing and colonoscopy. While colonoscopy remains the gold standard for the detection of colorectal lesions (up to 95% sensitivity for CRC and 76% for AA), compliance is not optimal owing to discomfort and unpleasant preparation procedures. The risk of complications, cost and access are other limitations of this procedure. On the other hand, the improved immunological version of fecal occult blood testing also referred to as the fecal immunochemical test (FIT), which detects human hemoglobin, has been used for some time with some success but poor precursor lesion detection rates (66-80% sensitivity for CRC but only 10-28% for AA) albeit an excellent specificity (93-95%) limits its effectiveness. It is therefore imperative to explore alternate or complementary strategies with the potential to improve CRC screening performance, especially for the detection of cancers at their early stages and AA.

In this context, a number of initiatives have been undertaken over the last ten years, from stool testing as a non-invasive approach to the implementation of personalized CRC screening trying to meet with desirable features for a CRC screening test. Interestingly, many of the stool-based testing strategies are based on the high rate of tumor cell exfoliation into the colon-rectal lumen, a parameter that appears to be independent of blood release. One of the best documented strategies is the FDA-approved multi-target stool DNA test, an approach based on the detection of specific DNA aberrations from the CRC cells shed into the stools in combination with FIT, which results in an improvement of sensitivity for both CRC (92.3%) and AA (42.4%) detection compared to FIT alone, although achieved through a reduction of specificity to 87% thus generating almost three times more false positives. At first sight, the cost-benefit of such new methods for the medical system may temper screening recommendations but the high cost of CRC treatment, particularly for more advanced disease, is considered to improve the cost-effectiveness of CRC screening. Furthermore, higher threshold costs for a biomarker test that could significantly increase the sensitivity of AA detection while maintaining reasonable specificity, would likely be cost-effective relative to currently available non-invasive tests.

Still based on the significant exfoliation of dysplastic cells from colorectal lesions into the lumen, host mRNA has also been investigated in the stools as a potential biomarker. While isolated from purified exfoliated colonocytes or directly extracted from the stools, host mRNA has been found to be a reliable source of biomarkers for detecting colorectal cancers. It was previously confirmed that the target mRNAs originated from the tumor or surrounding mucosa and that expression was affected by the number of exfoliated tumor cells, exfoliation of inflammatory cells, tumor size and transcript expression level in the tumor but not primary vs distal location. More recently, it has been demonstrated that the inclusion of a multi-target RNA assay significantly strengthens both sensitivity and specificity for CRC detection. Droplet digital PCR was also evaluated as a potential alternative to qPCR for stool mRNA multiplex analysis. However, one important question that remains to be tested for the validation of a multi-target stool mRNA test pertains to AA detection since, up to now, ITGA6 is the only target found to be overrepresented in stool samples of patients bearing AA. Another aspect that needs to be evaluated for potential clinical implementation is the robustness of the test under realistic preservation conditions, as mRNA are considered to be relatively susceptible to degradation in the stools.

It would be desirable to be provided with a non-invasive method to identify subjects who have an increased risk of having an advanced colorectal adenoma or a colorectal cancer with increased sensitivity and/or specificity.

### BRIEF SUMMARY

The present disclosure provides a method for stratifying subjects with respect to their relative risk of having an advanced colorectal adenoma or a colorectal cancer. The method is based on the differential expression of genes of colorectal epithelial cells which are present in the stool of the subject. The method is also based on the relative stability of mRNA transcripts in the stool.

According to a first aspect, the present disclosure concerns a method of stratifying the risk of a subject of having an advanced colorectal adenoma or a colorectal cancer in a subject. The method comprises a) providing a stool sample from the subject, wherein the stool sample comprises a plurality of mRNA transcripts from the subject. The method also comprises b) determining the mRNA expression level of at least two distinct genes from the plurality of mRNA transcripts to obtain a test expression profile. The method further comprises c) comparing the test expression profile with a control expression profile, wherein the control expression profile comprises the mRNA expression level of the at least two genes and is derived from a plurality of control mRNA transcripts (which can, in some embodiments, be derived from a control colorectal epithelial cell) from a control subject known to lack the advanced colorectal adenoma or the colorectal cancer. If it is determined that the test expression profile of the subject comprises at least two genes whose expression are increased with the respect to the control expression profile, the subject is stratified as having an increased risk of having the advanced colorectal adenoma or the colorectal cancer, when compared to the control subject. In some embodiments, the stool sample comprises at least one colorectal epithelial cell. In additional embodiments, the at least one colorectal epithelial cell comprises the plurality of mRNA transcripts. In yet additional embodiments, the test expression profile and the control expression profile comprise the mRNA expression level of at least two of the S100A4 gene, the GADD45B gene, the ITGA2 gene, the MYBL2 gene, the MYC gene, the CEACAM5 gene, and/or the MACC1 gene. In some specific embodiments, the present disclosure provides a method of stratifying the risk of a subject of having an advanced colorectal adenoma or a colorectal cancer in a subject, wherein the method comprises a) providing a stool sample from the subject, wherein the stool sample comprises at least one colorectal epithelial cell from the subject, b) determining the mRNA expression level of at least two distinct genes from the at least one colorectal epithelial cell to obtain a test expression profile, wherein the test expression profile comprises the mRNA expression level of at least two of the S100A4 gene, the GADD45B gene, the ITGA2 gene, the MYBL2 gene, the MYC gene, the CEACAM5 gene, and/or the MACC1 gene and c) comparing the test expression profile with a control expression profile, wherein the control expression profile comprises the mRNA expression level of the at least two genes and is derived from a control colorectal epithelial cell from a control subject known to lack the advanced colorectal adenoma or the colorectal cancer. In an embodiment, step b) comprises determining the mRNA expression level from at least one additional gene from plurality of mRNA transcripts or the colorectal epithelial cell, wherein the test expression profile and the control expression profile further comprises the expression level of the PTGS2 gene and/or of the ITGA6 gene. The method described herein can be used for stratifying the risk of the subject of having the advanced colorectal adenoma. In such embodiment, the test expression profile and the control expression profile can comprise the mRNA expression level of the CEACAM5 gene, the ITGA6 gene and/or the MACC1 gene. Alternatively or in combination, the method described herein can be used for stratifying the risk of the subject of having the colorectal cancer. In such embodiment, the test expression profile and the control expression profile can comprise the mRNA expression level of the S100A4 gene, the GADD45B gene, the ITGA2 gene, the MYBL2 gene, the MYC gene and/or the PTGS2 gene. In an embodiment, step b) comprises using a reverse-transcriptase polymerase chain reaction (RT-PCR) to obtain the mRNA expression level of the at least two genes of the test expression profile and/or the control expression profile. In yet another embodiment, step b) comprises using a quantitative polymerase chain reaction (qPCR) to obtain the mRNA expression level of the at least two gens of the test expression profile and/or the control expression profile. In some embodiments, the further comprises, prior to step b), storing the stool sample. In additional embodiment, the method further comprises determining the presence of hemoglobin in the stool sample. In some specific embodiments, the method comprises using a fecal immunochemical test (FIT) to determine the presence of hemoglobin in the stool sample. In further embodiments, the method further comprises determining the presence of a DNA mutation and/or an aberrant DNA methylation pattern associated with a predisposition to a colorectal cancer in the colorectal epithelial cell of the subject. For example, the DNA mutation can be located in the K-RAS gene. In another example, the aberrant DNA methylation pattern can be located in the NDRG4 gene and/or the BMP3 gene. In some embodiments, the method comprising using the Cologuard™ assay to determine the presence of hemoglobin in the stool sample, the presence of the DNA mutation and/or the presence of the aberrant DNA methylation pattern. In some embodiments, the method is for screening for subjects suitable for colonoscopy. In some embodiments, the method further comprises submitting the subject having been stratified as being at increased risk of developing the colorectal cancer to a chemotherapy, a radiotherapy and/or a surgery. In some embodiments, the colorectal cancer is a colon cancer or a rectal cancer.

According to a second aspect, the present disclosure provides a kit for stratifying the risk of a subject of having an advanced colorectal adenoma or a colorectal cancer in a subject, wherein the kit comprises at least two reagents for determining the mRNA expression level of at least two distinct genes from the plurality of mRNA transcripts to obtain a test expression profile in a stool sample from the subject. In some embodiments, the kit further comprises a container for storing a stool sample. In additional embodiments, the at least two reagents are for determining the mRNA expression level of the S100A4 gene, the GADD45B gene, the ITGA2 gene, the MYBL2 gene, the MYC gene, the CEACAM5 gene, and/or the MACC1 gene. In some additional embodiments, the kit further comprises at least one additional reagent for determining the mRNA expression level of the PTGS2 gene and/or of the ITGA6 gene. In yet additional embodiments, the at least two reagents are for determining the mRNA expression level of the CEACAM5 gene, the ITGA6 gene and/or the MACC1 gene. In still another embodiment, the at least two reagents are for determining the mRNA expression level of the S100A4 gene, the GADD45B gene, the ITGA2 gene, the MYBL2 gene, the MYC gene and/or the PTGS2 gene. In some embodiments, the kit further comprises a reverse-transcriptase. In still another embodiment, the kit can be used in combination or further means for determining the presence of hemoglobin in the stool sample. In still another embodiment, the kit can be used in combination or further comprises a fecal immunochemical test (FIT) to determine the presence of hemoglobin in the stool sample. In yet further embodiments, the kit can be used in combination or further comprises reagents for determining the presence of a DNA mutation and/or an aberrant DNA methylation pattern associated with a predisposition to a colorectal cancer in the colorectal epithelial cell of the subject. In some embodiments, the at least one DNA mutation is located in the K-RAS gene. In additional embodiments, the aberrant DNA methylation pattern is located in the NDRG4 gene and/or the BMP3 gene. In still some further embodiments, the kit can be used in combination or further comprises a Cologuard™ and/or a Coloalert™ assay to determine the presence of hemoglobin in the stool sample, the presence of DNA mutation and/or the presence of the abnormal DNA methylation pattern.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the detection and analysis of selected mRNA targets found to be overrepresented in stool samples of patients with colorectal cancer (CRC) stages I-III or advanced adenomas (AA). Results in A and B are expressed as median (interquartile range) of copy number and score, respectively, relative to control patients. ** P<0.001 to *** P<0.0005 using the Kruskal-Wallis test.

FIG. 1A (left panel) For S100A4, a significant increase was observed in CRC stages I-III as compared to controls (Ctrl) or patients with AA as one of the six targets identified as being overrepresented in the stools of patients with CRC. (right panel) For CEACAM5, a significant increase was observed in CRC stages I-III and AA as compared to controls (Ctrl) as one of the three targets identified as being overrepresented in the stools of patients with either AA or CRC.

FIG. 1B Scores were calculated using an algorithm that combined all six targets for CRC (left) and the three targets identifying AA and CRC (right) lesions relative to controls.

FIG. 1C Receiver operating characteristics (ROC) curve analysis showing the two groups of targets for CRC (left) and AA and CRC (right). Area under the curve (AUC) values are indicated.

FIG. 2 illustrates the ROC curve analysis of an optimized combination of five of the targets for the detection of patients with AA or CRC. AUC is indicated and sensitivity and specificity are provided in % (95% CI).

FIG. 2A ROC curve analysis of the combination of the three targets identified for detecting AA and CRC, CEACAM5, ITGA6 and MACC1 with the two stronger targets for detecting CRC, PTGS2 and S100A4, for AA and CRC.

FIG. 2B Same combination as in FIG. 2A but including the FIT component.

FIG. 3 provides the target stability analyses in stool samples over a 5-day period. Target stability was tested under various conditions of conservation and target detection was monitored throughout the 5 days in samples maintained at −20° C. with (F/T 5d −20) and without (5d −20° C.) a thaw cycle, at 4° C. (1-5d 4° C.) and at room temperature (1-5d RT).

FIG. 3A As illustrated with PTGS2, copy numbers remained relatively stable during the 5 days in both control stool samples (Ctrl) and samples obtained from CRC patients.

FIG. 3B Cumulative scores including the four tested targets PTGS2, CEACAM5, ITGA2 and ITGA6 showed that overall, the targets were relatively stable under cooled conditions and for 3 days at room temperature.

FIG. 4 provides the detection and analysis of selected mRNA targets found to be overrepresented in stool samples of patients with colorectal cancers (CRC) stages I-III or advanced adenomas (AA). As shown for S100A4 (FIG. 1A), a significant increase was observed for the five other targets GADD45B, ITGA2, MYBL2, MYC and PTGS2 in CRC stages IIII as compared to controls (Ctrl) while for three of the targets, CEACAM5 (FIG. 1A) ITGA6 and MACC1, a significant increase was observed in samples from patients with CRC stages I-III or AA as compared to controls (Ctrl). Results are expressed as median (interquartile range) of copy number relative to control patients. * P<0.05 to *** P<0.0005 using the Kruskal-Wallis test.

FIG. 4A Provides the copy number of GADD45B in function of the sample received.

FIG. 4B Provides the copy number of ITGA2 in function of the sample received.

FIG. 4C Provides the copy number of MYBL2 in function of the sample received.

FIG. 4D Provides the copy number of MYC in function of the sample received.

FIG. 4E Provides the copy number of PTGS2 in function of the sample received.

FIG. 4F Provides the copy number of ITGA6 in function of the sample received.

FIG. 4G Provides the copy number of MACC1 in function of the sample received.

FIG. 5 provides additional information on target stability analyses in stool samples over a 5-day period. Target stability was tested under various conditions of conservation as in FIG. 3 for CEACAM5, ITGA2 and ITGA6. Copy number (copy nb) were evaluated in the stool samples throughout the 5 days at −20OC with (F/T 5d −20) and without (5d −20) a thaw cycle, at 4° C. (1-5d 4) and at room temperature (1-5d RT).

FIG. 5A Provides the copy number of ITGA6 in control (left panel) or CRC (right panel) samples in function of days in storage.

FIG. 5B Provides the copy number of CEACAM5 in control (left panel) or CRC (right panel) samples in function of days in storage.

FIG. 5C Provides the copy number of ITGA2 in control (left panel) or CRC (right panel) samples in function of days in storage.

### DETAILED DESCRIPTION

The present disclosure provides a method for stratifying subjects with respect to their relative risk of having an advanced colorectal adenoma or a colorectal cancer by determining the expression levels of a plurality of genes in the subjects' stool sample. The subjects that can be stratified by the method can be mammals and, in some embodiments, humans. The subjects may or may not have been previously investigated for their predisposition to develop an advanced colorectal adenoma or a colorectal cancer. The subjects may or may not have been previously treated for an advanced colorectal adenoma or a colorectal cancer.

In some embodiments, when the method is performed to stratify the risk of having an advanced colorectal adenoma, it can have a sensitivity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher. In other embodiments, when the method is performed to stratify the risk of having a colorectal cancer, it can have a sensitivity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher.

Broadly, the method of the present disclosure allows the stratification of subjects into two groups: a first group of subjects having an increased risk of having an advanced colorectal adenoma or a colorectal cancer (e.g., high risk group) and a second group of subjects having a decreased risk of having an advanced adenoma or a colorectal cancer (e.g., low risk group). In some embodiments, the method can also allow the stratification of the high risk group into two subgroups: a first subgroup of subjects having an increased risk of having an advanced colorectal adenoma (e.g., advanced colorectal adenoma or AA subgroup) and a second subgroup of subjects having a decreased risk of having a colorectal cancer (e.g., colorectal cancer or CRC subgroup). The method is based on the overexpression of mRNA transcripts of at least two different genes present in the stool of the subjects. Subjects which have been stratified in the high risk group, the AA subgroup or the CRC subgroup can receive tailored recommendations and treatments. For example, subjects in the high risk group, especially in the CRC subgroup, can receive a recommendation to perform a colonoscopy and/or be subject to a colonoscopy. In another example, subjects in the high risk group, especially in the CRC subgroup, can receive a recommendation to receive a chemotherapy, a radiotherapy or to undergo surgery and/or receive the chemotherapy, the radiotherapy or be subject to surgery. Subjects which have been stratified in the low risk group can receive tailored recommendations and treatments.

The methods described herein rely on assessing the expression level of a combination of genes in one or more cells from the subject and determining if such genes are overexpressed in the stool sample obtained from the subjects. The mRNA transcripts which are being submitted to this method are present in a stool sample from the subject. It is understood that, in some embodiments, the mRNA transcripts can either be shed from cells of the colorectal epithelium and can be found in the stool sample in a cell-free manner. It also is understood that the mRNA transcripts can be present in one or more colorectal epithelial cell which is shed and present in the stool sample. The mRNA transcripts and/or the colorectal epithelial cell comprising same can be shed from an advanced colorectal adenoma(s) or a malignant epithelial tumor(s) that may be present in the subject. It has been surprisingly shown in the Example below that mRNA transcripts are stable in a stool sample and can conveniently be used to stratify the risk even though the stool sample had been previously stored.

As a first step, the method thus comprises providing a stool sample from the subject, wherein the stool sample comprises a plurality of mRNA transcripts from the colorectal epithelial cells from the subject. In one embodiment, the stool sample from the subject comprises at least one cell (or in some embodiments a plurality of cells) from the colorectal epithelium of the subject. In an embodiment, the cell is an epithelial cell. In still another embodiment, the cell is derived or shed from the colon's epithelium, e.g., the cell is a colon epithelial cell also referred to as a colonocyte. In yet another embodiment, the cell is derived or shed from the rectum's epithelium, e.g., the cell is a rectal epithelial cell. In still a further embodiment, the cell is derived or shed from the colon or the rectum, it is a colorectal epithelial cell. In some embodiments, the method comprises obtaining the stool sample of the subject.

In some embodiments, the method can be performed directly on the stool sample which has been obtained from the subject. In other embodiments, the method can be performed on a stool sample which has been processed. For example, the method can be performed on a stool sample which has been diluted with an appropriate solution (which can, in some embodiments, include RNase inhibitors) and/or filtered. As such, the method can include, in some embodiments, diluting and/or filtering the stool sample.

In yet another example, the stool sample or the processed stool sample can be stored prior to the next (e.g., determining) step. The stool sample or the processed stool sample can be stored at freezing temperatures (e.g., between −25° C. and −15° C., in some embodiments at −18° C.), at refrigerating temperatures (e.g., between 0° C. and 10° C., in some embodiments at 4° C.) and/or at room temperatures (e.g., between 20° C. and 30° C., in some embodiments at 23° C.). As such, the method can include storing the stool sample or the processed stool sample after it has been obtained or processed and before it is being further characterized. The stool sample or the processed stool sample can be stored for at least 1, 2, 3, 4, 5 days or more prior to the determination of the mRNA expression levels. In some embodiments, the method can include storing the stool sample or the processed stool sample prior to determining the mRNA expression levels.

Once the stool sample (which may have been processed and/or stored) has been obtained, the expression level of at least two distinct genes from the one or more cell present in the stool sample is being determined. The expression level of the at least two distinct genes are obtaining by determining the (relative) amount of the mRNA being expressed from each genes. The determination of the expression level of the combination of genes can be made simultaneously (in a multiplex format) or subsequently. The determination of the expression level of the combination of genes can include the reverse transcription of the mRNA transcripts associated with each gene, the amplification of the cDNA molecules associated with each gene of the combination and/or the hybridization of an oligonucleotide (which may be a primer or a probe) to the mRNA transcripts/cDNA molecules associated with each gene of the combination. In embodiments of the methods in which the mRNA transcripts are being reverse-transcribed and amplified, their (relative) amount can be determined by detecting and optionally quantified a signal associate to the amplified nucleic acid molecules. In some embodiments, the method can include performing a reverse-transcription step to convert the mRNA transcripts into cDNA molecules. In some additional embodiments, the method can include performing a polymerase chain reaction (PCR) step to amplify the number of cDNA molecules. In yet further embodiments, the method can include performing a quantitative polymerase chain reaction (qPCR) step to quantify the number of cDNA molecules. In yet further embodiments, the method can include performing a digital polymerase chain reaction (dPCR) step to quantify the number of cDNA molecules. In some embodiments, the mRNA expression level can be provided as an absolute amount or can be provided in a normalized amount (for example for an amount respective to the number or cells or another mRNA transcript or combination of mRNA transcripts whose expression is known not to be modulated in advanced colorectal adenoma or colorectal cancer cells). In some embodiments in which the method provides a normalized amount, the method can further include determining the number of cells in which the mRNA expression level has been determined and/or determining the mRNA expression level of one or more household gene in the stool sample or the processed stool sample. In some embodiments, the mRNA expression levels can be provided as ratios of one another.

The determination step provides a test expression profile which comprises the mRNA expression level of the at least two genes whose expression has been quantified. The test expression profile can include the mRNA expression level of the CEA adhesion molecule 5 (also referred to as CEACAM5, CD66e or CEA and having the Gene ID 1048). The test expression profile can include the mRNA expression level of the growth arrest and DNA damage inducible beta gene (also referred to as GADD45B, GADD45BETA or MYD118 and having the Gene ID: 4616). The test expression profile can include the mRNA expression level of the integrin subunit alpha 2 gene (also referred to as ITGA2, BR, CD49B, GPIa, HPA-5, VLA-2 or VLAA2 and having the Gene ID: 3673). The test expression profile can include the mRNA expression level of the MET transcriptional regulator MACC1 (also referred to as MACC1, 7A5 or SH3BP4L and having the Gene ID: 346389). The test expression profile can include the mRNA expression level of the MYB proto-oncogene like 2 gene (also referred to as MYBL2, B-MYB or BMYB and having the Gene ID: 4605). The test expression profile can include the mRNA expression level of the MYC proto-oncogene, bHLH transcription factor (also referred to as MYC, MRTL, MYCC, bHLHe39 or c-Myc and having the Gene ID: 4609). The test expression profile can include the mRNA expression level of the S100 calcium binding protein A4 (also referred to as S100A4, 18A2, 42A, CAPL, FSP1, MTS1, P9KA or PEL98 and having Gene ID: 6275). In some further optional embodiments, the test expression profile can include the mRNA expression level of the beta-2-microglobulin gene (also referred to as B2M or IMD43 and having the Gene ID 567). In some further optional embodiments, the test expression profile can include the mRNA expression level of the integrin subunit alpha 1 gene (also referred to as ITGA1, CD49a or VLA1 and having the Gene ID: 3672). In a specific embodiment, the test expression profile can include the mRNA expression profile of CEACAM5, ITGA6 and MACC1. In yet another specific embodiment, the test expression profile can include the mRNA expression profile of CEACAM5, ITGA6, MACC1 and B2M. In still yet another embodiment, the test expression profile can include the mRNA expression profile of PTGS2 and S100A4. In still yet another embodiment, the test expression profile can include the mRNA expression profile of CEACAM5, ITGA6, MACC1, PTGS2 and S100A4, optionally in combination with the mRNA expression profile of B2M.

In some optional embodiments, the test expression profile can include the mRNA expression level of the integrin subunit alpha 6 gene (also referred to as ITGA6, CD49f, ITGA6B or VLA-6 and having the Gene ID: 3655). In such embodiments, it is possible that the test expression profile can include the mRNA expression level of the alpha- and/or beta-isoform of ITAGA6 gene transcript. In some optional embodiments, the test expression profile can include the mRNA expression level of the prostaglandin-endoperoxide synthase 2 gene (also referred to as PTGS2, COX-2, COX2, GRIPGHS, PGG/HS, PGHS-2, PHS-2 or hCox-2 and having the Gene ID: 5743).

In some specific embodiments, the test expression level can include at least one, two, three, four or five level of mRNA expression the S100A4 gene, the GADD45B gene, the ITGA2 gene, the MYBL2 gene, the MYC gene and/or the PTGS2 gene.

The test expression profile comprises the mRNA expression level of a combination of at least two of any of the genes described herein. In some embodiments, the test expression profile comprises the mRNA expression level of a combination of at least three of any of the genes described herein. In some embodiments, the test expression profile comprises the mRNA expression level of a combination of at least four of any of the genes described herein. In some embodiments, the test expression profile comprises the mRNA expression level of a combination of at least five of any of the genes described herein. In some embodiments, the test expression profile comprises the mRNA expression level of a combination of at least six of any of the genes described herein. In some embodiments, the test expression profile comprises the mRNA expression level of a combination of at least seven of any of the genes described herein. In some embodiments, the test expression profile comprises the mRNA expression level of a combination of at least eight of any of the genes described herein. In some embodiments, the test expression profile comprises the mRNA expression level of a combination of at least nine of any of the genes described herein.

In some embodiments, the test expression profile comprises the mRNA expression level of a combination of at least ten of any of the genes described herein. In some embodiments, the test expression profile comprises the mRNA expression level of a combination of at least eleven of any of the genes described herein. In some embodiments, the test expression profile comprises the mRNA expression level of a combination of at least twelve of any of the genes described herein.

Once the test expression profile has been obtained, it is compared to a control expression profile. The control expression profile comprises the mRNA expression level of the at least two genes who are present on the test expression level. The control expression profile can be obtained or derived from one or more mRNA transcripts and/or cells (such as, for example, one or more epithelial cells and, in further embodiments, one or more colorectal epithelial cell) from a control subject which is known not to experience an advanced colorectal adenoma or a colorectal cancer (e.g., a healthy control subject). The control expression profile can be obtained or derived from one or more mRNA transcripts and/or cells from a control subject having a non-advanced colorectal adenoma and lacking an advanced colorectal adenoma or a colorectal cancer. The control expression profile can be obtained or derived from one or more RNA transcripts and/or one or more cells from a healthy (e.g., non cancerous) tissue from a subject which may, in some embodiments, have an advanced colorectal adenoma or a colorectal cancer. In some embodiments, the control subject can be aged- and gender-matched with the subject whose risk is being stratified. In some embodiments, the control expression profile is obtained or derived from a plurality of control subjects. In some further embodiments, the method can include determining the mRNA expression profile of at least two genes from the control subject to provide the control expression profile.

The control expression profile can include the mRNA expression level of the CEA adhesion molecule 5 (also referred to as CEACAM5, CD66e or CEA and having the Gene ID 1048). The control expression profile can include the mRNA expression level of the growth arrest and DNA damage inducible beta gene (also referred to as GADD45B, GADD45BETA or MYD118 and having the Gene ID: 4616). The control expression profile can include the mRNA expression level of the integrin subunit alpha 2 gene (also referred to as ITGA2, BR, CD49B, GPIa, HPA-5, VLA-2 or VLAA2 and having the Gene ID: 3673). The control expression profile can include the mRNA expression level of the MET transcriptional regulator MACC1 (also referred to as MACC1, 7A5 or SH3BP4L and having the Gene ID: 346389). The control expression profile can include the mRNA expression level of the MYB proto-oncogene like 2 gene (also referred to as MYBL2, B-MYB or BMYB and having the Gene ID: 4605). The control expression profile can include the mRNA expression level of the MYC proto-oncogene, bHLH transcription factor (also referred to as MYC, MRTL, MYCC, bHLHe39 or c-Myc and having the Gene ID: 4609). The control expression profile can include the mRNA expression level of the S100 calcium binding protein A4 (also referred to as S100A4, 18A2, 42A, CAPL, FSP1, MTS1, P9KA or PEL98 and having Gene ID: 6275). In a specific embodiment, the control expression profile can include the mRNA expression profile of CEACAM5, ITGA6 and MACC1. In yet another specific embodiment, the control expression profile can include the mRNA expression profile of CEACAM5, ITGA6, MACC1 and B2M. In still yet another embodiment, the control expression profile can include the mRNA expression profile of PTGS2 and S100A4. In still yet another embodiment, the control expression profile can include the mRNA expression profile of CEACAM5, ITGA6, MACC1, PTGS2 and S100A4, optionally in combination with the mRNA expression profile of B2M.

In some optional embodiments, the control expression profile can include the mRNA expression level of the integrin subunit alpha 6 gene (also referred to as ITGA6, CD49f, ITGA6B or VLA-6 and having the Gene ID: 3655). In such embodiments, it is possible that the control expression profile can include the mRNA expression level of the alpha- and/or beta-isoform of ITAGA6 gene transcript. In some optional embodiments, the control expression profile can include the mRNA expression level of the prostaglandin-endoperoxide synthase 2 gene (also referred to as PTGS2, COX-2, COX2, GRIPGHS, PGG/HS, PGHS-2, PHS-2 or hCox-2 and having the Gene ID: 5743). In some further optional embodiments, the control expression profile can include the mRNA expression level of the beta-2-microglobulin gene (also referred to as B2M or IMD43 and having the Gene ID 567). In some further optional embodiments, the control expression profile can include the mRNA expression level of the integrin subunit alpha 1 gene (also referred to as ITGA1, CD49a or VLA1 and having the Gene ID: 3672).

In some specific embodiments, the control expression level can include at least one, two, three, four or five level of mRNA expression the S100A4 gene, the GADD45B gene, the ITGA2 gene, the MYBL2 gene, the MYC gene and/or the PTGS2 gene.

The control expression profile comprises the mRNA expression level of the genes which are also reported on the test expression profile. In some embodiments, the control expression profile comprises the mRNA expression level of a combination of at least two of any of the genes described herein. In some embodiments, the control expression profile comprises the mRNA expression level of a combination of at least three of any of the genes described herein. In some embodiments, the control expression profile comprises the mRNA expression level of a combination of at least four of any of the genes described herein. In some embodiments, the control expression profile comprises the mRNA expression level of a combination of at least five of any of the genes described herein. In some embodiments, the control expression profile comprises the mRNA expression level of a combination of at least six of any of the genes described herein. In some embodiments, the control expression profile comprises the mRNA expression level of a combination of at least seven of any of the genes described herein. In some embodiments, the control expression profile comprises the mRNA expression level of a combination of at least eight of any of the genes described herein. In some embodiments, the control expression profile comprises the mRNA expression level of a combination of at least nine of any of the genes described herein. In some embodiments, the control expression profile comprises the mRNA expression level of a combination of at least ten of any of the genes described herein. In some embodiments, the control expression profile comprises the mRNA expression level of a combination of at least eleven of any of the genes described herein. In some embodiments, the control expression profile comprises the mRNA expression level of a combination of at least twelve of any of the genes described herein.

A comparison is then conducted to determine if the mRNA expression levels present in the test expression profile are higher than the corresponding mRNA expression levels present in the control expression profile. This comparison is performed on a gene-by-gene basis. For example, if the test expression profile comprises the mRNA expression level of the CEACAM5 gene, such expression level is compared to the mRNA expression level of the CEACAM5 in the control expression profile. If it is determined that the mRNA expression levels of at least two genes in the test expression profile is higher than the mRNA expression levels of the same two genes in the control expression profile, this is indicative that the stratified subject has an increased risk of having an advanced adenoma or a colorectal cancer when compared to the control subject. If it is determined that the mRNA expression levels of at least two genes in the control expression profile is lower than the mRNA expression levels of the same two genes in the test expression profile, this is indicative that the stratified subject has an increased risk of having an advanced colorectal adenoma or a colorectal cancer when compared to the control subject.

As indicated above, the methods described herein can also be used to stratify the risk of the subject of having an advanced colorectal adenoma. In such embodiments, the test and control expression profiles include the mRNA expression levels of the CEACAM5 gene, the ITGA6 gene and/or the MACC1 gene. In some additional embodiments, the test and control expression profiles include the mRNA expression levels of the CEACAM5 gene, the ITGA6 gene and the MACC1 gene. The method can include determining the mRNA expression level of any one of the CEACAM5 gene, the ITGA6 gene and/or the MACC1 gene in the subject being stratified and/or the control subject. In some specific embodiments, the method can include determining the mRNA expression level of the CEACAM5 gene, the ITGA6 gene and the MACC1 gene in the subject being stratified and/or the control subject. The method can include comparing the mRNA expression level of the CEACAM5 gene, the ITGA6 gene and/or the MACC1 gene between the test and the control expression profiles. In some embodiments, the method can include comparing the mRNA expression level of the CEACAM5 gene, the ITGA6 gene and the MACC1 gene between the test and the control expression profiles. If it has been determined that the mRNA expression level of at least one, at least two or all three genes (e.g., the CEACAM5 gene, the ITGA6 gene and/or the MACC1 gene) present in the test expression profile is increased with respect the corresponding mRNA expression level in the control expression profile, it is indicative that the stratified subject has an increased risk, with respect to the control subject, of having an advanced colorectal adenoma. If it has been determined that the mRNA expression level of at least one, at least two or all three genes (e.g., the CEACAM5 gene, the ITGA6 gene and/or the MACC1 gene) present in the control expression profile is decreased with respect the corresponding mRNA expression level in the test expression profile, it is indicative that the stratified subject has an increased risk, with respect to the control subject, of having an advanced colorectal adenoma.

As indicated above, the methods described herein can also be used to stratify the risk of the subject of having a colorectal cancer. In such embodiments, the test and control expression profiles include the mRNA expression levels of the S100A4 gene, the GADD45B gene, the ITGA2 gene, the MYBL2 gene, the MYC gene and/or the PTGS2 gene. In some additional embodiments, the test and control expression profiles include the mRNA expression levels of the S100A4 gene, the GADD45B gene, the ITGA2 gene, the MYBL2 gene, the MYC gene and the PTGS2 gene. The method can include determining the mRNA expression level of any one of the S100A4 gene, the GADD45B gene, the ITGA2 gene, the MYBL2 gene, the MYC gene and/or the PTGS2 gene in the subject being stratified and/or the control subject. In some specific embodiments, the method can include determining the mRNA expression level of the S100A4 gene, the GADD45B gene, the ITGA2 gene, the MYBL2 gene, the MYC gene and the PTGS2 gene in the subject being stratified and/or the control subject. The method can include comparing the mRNA expression level of the S100A4 gene, the GADD45B gene, the ITGA2 gene, the MYBL2 gene, the MYC gene and/or the PTGS2 gene between the test and the control expression profiles. In some embodiments, the method can include comparing the mRNA expression level of the S100A4 gene, the GADD45B gene, the ITGA2 gene, the MYBL2 gene, the MYC gene and/or the PTGS2 gene between the test and the control expression profiles. If it has been determined that the mRNA expression level of at least one, at least two, at least three, at least four or all five genes (e.g., the S100A4 gene, the GADD45B gene, the ITGA2 gene, the MYBL2 gene, the MYC gene and/or the PTGS2 gene) present in the test expression profile is increased with respect the corresponding mRNA expression level in the control expression profile, it is indicative that the stratified subject has an increased risk, with respect to the control subject, of having a colorectal cancer. If it has been determined that the mRNA expression level of at least one, at least two, at least three, at least four or all five genes (e.g., the S100A4 gene, the GADD45B gene, the ITGA2 gene, the MYBL2 gene, the MYC gene and/or the PTGS2 gene) present in the control expression profile is decreased with respect the corresponding mRNA expression level in the test expression profile, it is indicative that the stratified subject has an increased risk, with respect to the control subject, of having a colorectal cancer.

The stratification methods described herein can be used in conjunction with other methods and assays used to aid the diagnosis of advanced colorectal adenoma or colorectal cancer. It is recognized in the art that the presence of hemoglobin, a component of blood, in the stool is present more frequently in subjects having an advanced colorectal adenoma or a colorectal cancer than in subjects having a non-advanced adenoma or healthy subjects. As such, the stratification methods described herein can be used in combination with the determination of the presence of hemoglobin to increase the sensitivity of the methods. The methods described herein can thus include a step of determining the presence of hemoglobin in the stool sample or the processed stool sample. In some embodiments, the method can include performing a fecal immunochemical test (FIT) to determine the presence or absence of hemoglobin in the stool sample. In some further embodiments, the method can include performing a guaic-based fecal occult blood test (gFOBT). In some additional embodiments, the method can include performing the Cologuard™ and/or the ColoAlert™ test. One of the strength of combining the method of th present disclosure based on multitarget mRNA with other assays is the higher level of detection of advanced adenomas with mRNA targets as compared to other non invasive methods including FIT or gFOBT, Cologuard™ and/or ColoAlert™.

The subject being stratified may have previously been determined with the presence of an advanced adenoma or a colorectal cancer. For example, the presence of hemoglobin in the stool of the subject being stratified may have previously been determined. In some embodiments, the subject being stratified may previously have been submitted to a FIT or a gFOBT tests to determine the presence of hemoglobin in a stool sample (which may be the same or different from the one used to determine the mRNA expression levels). In some embodiments, the subject being stratified may previously had been determined to have hemoglobin in his/her stool.

It is also recognized that some DNA mutations increase the predisposition of a subject to develop an advanced adenoma or a colorectal cancer (when compared to a control subject). As such, the stratification methods described herein can be used in combination with the determination of the presence or the absence of one or more DNA mutation in the genome of cells of the subjects to increase the sensitivity of the methods described herein. The methods described herein can thus include a step of determining the presence or absence of at least one DNA mutation (which may be, for example, a deletion, an insertion and/or a duplication) in the genome of the subjects, wherein the at least one DNA mutation is associated with an increase in the predisposition of developing an advanced adenoma or a colorectal cancer. For example, the DNA mutation can be located in the NDRG4 gene, the BMP3 gene and/or the K-RAS gene. In some embodiments, the method can include performing the Cologuard™ test to determine the presence of the at least one DNA mutation in the cells of the subject.

The subject being stratified may have previously been diagnosed with the presence of an advanced adenoma or a colorectal cancer. For example, the presence of at least one DNA mutation in the cell(s) of the subject being stratified may have previously been determined. In some embodiments, the subject being stratified may previously have been submitted to a Cologuard™ test to determine the presence of the DNA mutation in the cell from the subject.

It is also recognized that advanced adenoma and malignant colorectal tumors can be visualized in situ and help the physician in determining if a subject has an advanced colorectal adenoma or a colorectal cancer. As such, the stratification methods described herein can be used in combination with the visualization of part of the subject's colorectal tract. The colorectal tract can be visualized using a colonoscopy, a flexible sigmoidoscopy and/or a CT colonography. In some embodiments, the colorectal tract can be visualized using a colonoscopy. The methods described herein can thus include a step of visualizing part or the entire subject's colorectal tract to determine the presence of advanced colorectal adenoma(s) and/or malignant colorectal tumor(s). In some embodiments, the subject being stratified may have previously been submitted to a visualization of part or all of its colorectal tract.

Alternatively, the methods described herein can be used prior to the visualization of the subject's colorectal tract. For example, the methods described herein can be used to prioritize subjects being stratified in the high risk group or the CRC subgroup of being submitted to imaging, such as a colonoscopy. Imaging techniques are uncomfortable for some subjects or can be of limited availability in some geographical areas. As such, there may be, under certain circumstances, a need to prioritize subjects which would benefit from such imaging analysis because they are in the high risk group or the CRC subgroup. In some embodiments, the method include recommending to the subject having been stratified in the high risk group or the CRC subgroup of having an imaging analysis of their colorectal tract (such as a colonoscopy) performed to aid the physician in his/her diagnosis.

The methods described herein can also be used in the context of a clinical trial to include or exclude subjects from a clinical study or to attribute them to a treatment arm of the clinical study.

It is also recognized that advanced adenoma and malignant colorectal tumors can be detected in a pathology analysis (such as, for example, an histology analysis) and help the physician in determining if a subject has an advanced colorectal adenoma or a colorectal cancer. As such, the stratification methods described herein can be used in combination with a pathological analysis of a tissue of a subject suspected of being an advanced colorectal adenoma or a malignant colorectal tumor. In some embodiments, the tissue of the subject being stratified may have previously been submitted to pathological analysis.

Once stratified to a particular group or a particular subgroup, the subject may receive a tailored therapeutic regimen that is suitable to alleviate the symptoms or treat the condition that subject has been assigned to. As such, the methods described herein can be used in the treatment of an advanced colorectal adenoma or a colorectal cancer (such as a colon cancer or a rectal cancer) in subjects which have been stratified in the high risk group. The treatment can include submitting the subject to one or more rounds of chemotherapy (e.g., 5-fluorouracil, leucovorin, capacitabine, irinotecan and/or oxaliplatin) optionally in combination with therapeutic antibodies. The treatment can include submitting the subject to one or more rounds of radiation therapy. The treatment can include submitted the subject to surgery (e.g., surgical resection of the advanced colorectal adenoma or the malignant colorectal tumor).

The methods described herein can be used to tailor the treatment regimen of a subject which has received at least one dose of chemotherapy, at least one dose or radiotherapy and/or has already been submitted to a surgery to remove one or more advanced colorectal adenoma or one or more colorectal malignant tumor. In such embodiments, the methods can be used to determine if the subject is at risk of having pre-cancerous or cancerous colorectal cells or if the treatment provided was sufficient to reduce the risk of having pre-cancerous or cancerous colorectal cells. As such, the methods described herein can be used after the subject has received at least one first therapy or surgery and before the subject received a further therapy or is submitted to a further surgery. The methods described herein can help the physician to determine if a more aggressive or a less aggressive therapeutic or surgical regimen is required.

The present disclosure also provides a kit for performing the stratification methods. The kit comprises means (e.g., reagents) for determining the mRNA expression levels of the at least one, two, three, four, five or more genes present on the test expression profile. For example, the kit can comprise at least one, two, three, four, five or more pair of primers for amplifying the cDNA molecules corresponding to the mRNA molecules whose expression is being determined. The kit can include reagents for the detection of the mRNA expression level of the CEA adhesion molecule 5 (also referred to as CEACAM5, CD66e or CEA and having the Gene ID 1048). The kit can include reagents for the detection the mRNA expression level of the growth arrest and DNA damage inducible beta gene (also referred to as GADD45B, GADD45BETA or MYD118 and having the Gene ID: 4616). The kit can include reagents for the dectection of the mRNA expression level of the integrin subunit alpha 2 gene (also referred to as ITGA2, BR, CD49B, GPIa, HPA-5, VLA-2 or VLAA2 and having the Gene ID: 3673). The kit can include reagents for the detection of the mRNA expression level of the MET transcriptional regulator MACC1 (also referred to as MACC1, 7A5 or SH3BP4L and having the Gene ID: 346389). The test expression profile can include the mRNA expression level of the MYB proto-oncogene like 2 gene (also referred to as MYBL2, B-MYB or BMYB and having the Gene ID: 4605). The kit profile can include reagents for the detection of the mRNA expression level of the MYC proto-oncogene, bHLH transcription factor (also referred to as MYC, MRTL, MYCC, bHLHe39 or c-Myc and having the Gene ID: 4609). The kit can include reagents for the detection of the mRNA expression level of the S100 calcium binding protein A4 (also referred to as S100A4, 18A2, 42A, CAPL, FSP1, MTS1, P9KA or PEL98 and having Gene ID: 6275). In some further optional embodiments, the kit can include the reagents for the detection of the mRNA expression level of the beta-2-microglobulin gene (also referred to as B2M or IMD43 and having the Gene ID 567). In some further optional embodiments, the kit can include reagents for the detection of the mRNA expression level of the integrin subunit alpha 1 gene (also referred to as ITGA1, CD49a or VLA1 and having the Gene ID: 3672). In a specific embodiment, the kit can include reagents for the detection of the mRNA expression profile of CEACAM5, ITGA6 and MACC1. In yet another specific embodiment, the test expression profile can include the mRNA expression profile of CEACAM5, ITGA6, MACC1 and B2M. In still yet another embodiment, the kit can include reagents for the detection of the mRNA expression profile of PTGS2 and S100A4. In still yet another embodiment, the kit can include reagents for the detection of the mRNA expression profile of CEACAM5, ITGA6, MACC1, PTGS2 and S100A4, optionally in combination with the mRNA expression profile of B2M. In some optional embodiments, the kit can include reagents for the detection of the mRNA expression level of the integrin subunit alpha 6 gene (also referred to as ITGA6, CD49f, ITGA6B or VLA-6 and having the Gene ID: 3655). In such embodiments, it is possible that the kit can include reagents for the detection of the expression of the mRNA expression level of the alpha- and/or beta-isoform of ITAGA6 gene transcript. In some optional embodiments, kit can include reagents for the detection of the mRNA expression level of the prostaglandin-endoperoxide synthase 2 gene (also referred to as PTGS2, COX-2, COX2, GRIPGHS, PGG/HS, PGHS-2, PHS-2 or hCox-2 and having the Gene ID: 5743). In some specific embodiments, the kit can include at least one, two, three, four or five reagents for the detection of the level of expression the S100A4 gene, the GADD45B gene, the ITGA2 gene, the MYBL2 gene, the MYC gene and/or the PTGS2 gene.

The kit can, in some embodiments, include a polymerase for performing a polymerase chain reaction. In some embodiments, the kit can also include primers for performing the reverse-transcription step. The kit can, in some additional embodiments, include a reverse transcriptase for performing the reverse transcription step. In some embodiments, the kit can include probes intended to be cleaved during the amplification step (e.g., Taqman® probes for example) to allow a quantitative PCR detection of the mRNA transcripts. The kit can also include a container for a stool sample from the subject and/or for storing the stool sample prior to the determination step. The kit also comprises instructions to use the means for determining the mRNA expression levels to obtain the test expression profile. The kit can also include instructions on how to stratify the subjects whose stool sample is being analyzed based on their risk of having an advanced adenoma or a colorectal cancer.

The present invention will be more readily understood by referring to the following examples which are given to illustrate the invention rather than to limit its scope.

### Example

Patients and samples. Two sets of patient samples were used. The first set of samples was collected from patients and healthy controls from the Hamamatsu University School of Medicine with written informed consent. The study was approved by the institutional research ethics committee of the Hamamatsu University School of Medicine. Complete information about this set has been provided in previous studies (Herring et al., 2018; Beaulieu et al., 2016; Herring et al., 2017). Briefly, the study cohort used herein included 24 patients with AA defined as being 10 mm or larger at their greatest dimension and 78 patients with CRC (24 stage I, 32 stage II and 22 stage III) diagnosed by colonoscopy and histopathology as well as 32 healthy controls. For controls and AA, stool samples were collected before colonoscopy. The immunochemical fecal occult blood test (iFOBT) was performed on all patients and controls as described (Beaulieu et al., 2016).

The second set of samples was collected from three healthy controls and three patients diagnosed with CRC stage II or III by colonoscopy and histopathology from the Centre Hospitalier Universitaire de Sherbrooke (CHUS) with written informed consent. The study was approved by the institutional research ethics committee of the CHUS. This set of samples was used for mRNA target stability experiments. Each sample was split into 13 aliquots stored under various conditions for up to 5 days as follows: #1, 5 days at −80° C. used as control; #2, 5 days at −20° C.; #3, 5 days at −20° C. with a thaw/freeze cycle; #4-8, 1-5 days at 4° C. and #9-13, 1-5 days at 23° C.

RNA isolation, reverse transcription, preamplification, and PCR amplification. RNA was isolated from fecal samples and reverse transcribed as described previously (Hamaya et al., 2010; Dydensborg et al., 2006). For preamplification, the TaqMan PreAmp Master Kit (Life Technology) was used to provide unbiased, multiplex preamplification of specific amplicons for analysis with TaqMan gene expression assays (Herring et al., 2017). Commercially available TaqMan primer and probe mixtures were used for the preamplification of the 27 preselected targets as described before 30 and detailed in Table 1. Quantitative polymerase chain reaction (qPCR) was performed using the TaqMan Gene Expression Assay with conditions described previously (Herring et al., 2018).

TABLE 1
List of specific targets tested. All primer and probe mixtures were
first tested on a subset of stool samples including controls, AA
and CRC to select those that were consistently detectable in the
stools. Further analysis on the whole set of samples allowed the
selection of those specifically enriched in CRC and AA or only CRC.

| | | Consistently | Over-represented | |
|---|---|---|---|---|
| | | Detected in | CRC | AA and |
| Gene name | TaqMan Asay I.D. | stools | only | CRC |
| B2M | Hs00984230_m1 | Y | | Y |
| BGN | Hs00156076_m1 | | | |
| CEACAM5 | Hs00944025_m1 | Y | | Y |
| CTNNB1 | Hs00355049_m1 | | | |
| DYNC2H1 | Hs00941787_m1 | | | |
| FAP | Hs00990806_m1 | | | |
| GADD45B | Hs00169587_m1 | Y | Y | |
| GLI1 | Hs00171790_m1 | | | |
| HMAN1B1 | Hs01032463_m1 | | | |
| HNRNPA2B1 | Hs00955384_m1 | | | |
| INHBA | Hs04187260_m1 | | | |
| ITGA1 | Hs00235006_m1 | Y | | |
| ITGA2 | Hs01673848_m1 | Y | | |
| ITGA6A | Hs01041013_m1 | Y | Y | |
| ITGA6 | Hs01041011_m1 | Y | | Y |
| KI67 | Hs01032434_m1 | | | |
| KIF3A | Hs01126351_m1 | | | |
| KIF7 | Hs00419527_m1 | | | |
| MACC1 | Hs00766186_m1 | Y | | Y |
| MLH1 | Hs00179866_m1 | Y | | |
| MSH1 | Hs00954125_m1 | Y | | |
| MTR | Hs01090031_m1 | | | |
| MYBL2 | Hs00942543_m1 | Y | Y | |
| MYC | Hs00153408_m1 | Y | Y | |
| PTGS2 | Hs00153133_m1 | Y | Y | |
| S100A4 | Hs00243202_m1 | Y | Y | |
| VDAC2 | Hs01075603_m1 | | | |

Data presentation and statistical analysis. Stool mRNA data were calculated as copy number per μl of reaction. For each transcript, a standard reference curve was generated using a serial fivefold dilution of a cDNA stock solution of the target sequence quantified on a NanoDrop 1000 Spectrophotometer (NanoDrop, Wilmington, DE, USA). Prism 8 was used for calculating statistics. Comparison mRNA expression (in copy number) in stool controls and patients with AA and CRC stage I-III lesions were expressed as median with interquartile range and analyzed by the Kruskal-Wallis test followed by Dunn's multiple comparison test. Area under the receiver operating characteristic (ROC) curves were calculated to establish sensitivities and specificities for each marker expressed in % with a 95% confidence interval. Scores were calculated for each marker on a scale of 0 to 3 on the basis of three cut-off values established from the ROC curve: (the lower cut-off corresponding to a sensitivity of 80%, medium cut-off corresponding to a specificity of 90% and higher cut-off corresponding to a specificity of 99%) as established previously. 29 Statistical significance was defined as P<0.05.

Twenty-seven (27) specific targets chosen on the basis of their reported over expression in colorectal cancerous lesions were screened. Preliminary evaluation of these using a subset of 30 samples (10 controls, 10 AA and 10 CRC) revealed that 14 were consistently detected in stools of patients bearing colorectal lesions (Table 1). Further testing with other primer and probe mixtures for poorly detected targets was tried but not further studied herein since 14 appeared to be enough to run the validation assay considering that for a clinical assay, the multiplex PCR capacity is limited to 4 to 5 targets depending on the manufacturer.

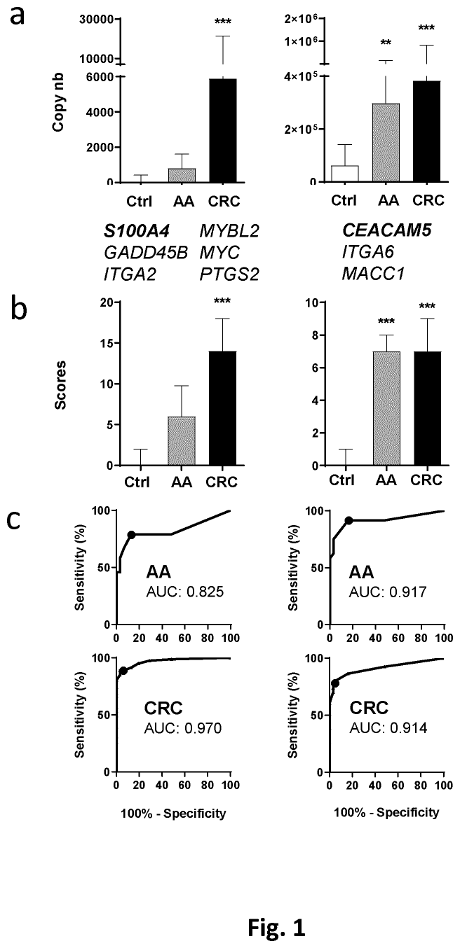

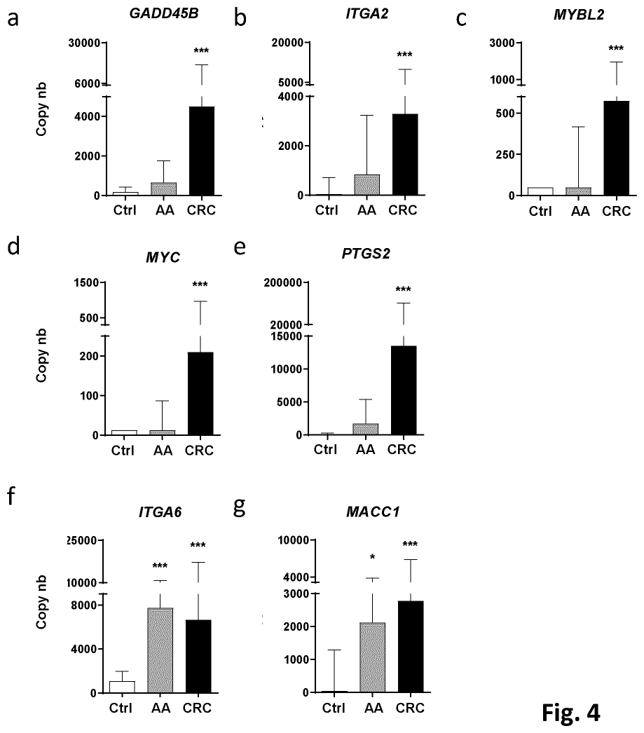

Further investigation of the 14 targets was performed on the set of 132 samples obtained from healthy controls (32) and patients bearing colorectal lesions (24 AA and 78 CRC). As shown in Table 1, a number of targets were found to be significantly over-represented in samples from patients with CRC while a few identified patients bearing AA or CRC. As illustrated with S100A4 (FIG. 1A), the median copy number for the transcripts of the first group which also included GADD45B, ITGA2, MYBL2, MYC and PTGS2 (FIG. 4) were found to be significantly increased in the stools of patients with CRC as compared with the controls while only three including CEACAM5 (FIG. 1A), ITGA6 and MACC1 (FIG. 4) were found to be over-represented also in patients with AA.

Scores were then calculated for the two groups of markers. Because copy numbers varied considerably between the targets, from −200 for MYC to 40,000 for CEACAM5, individual scores were determined for all targets by attributing a value of 0 to 3 for each patient sample based the on cut-off values of the targets, as described above. Then, an overall score for the each of the two groups of markers was determined for controls and patients with AA or CRC. As shown in FIG. 1B, the overall score for the 6 markers of the first group significantly recognized the samples from CRC patients vs those of the controls while the overall scores of the three markers of the second group distinguished the samples from patients bearing CRC or AA from those of the controls. ROC curves for the two groups were determined (FIG. 1C). For the first group, the area under the curve (AUC) for CRC was 0.970 corresponding to a sensitivity of 89% for 95% specificity but AUC was only 0.825 for AA with a 58% sensitivity (for 95% specificity). In the second group, AUC was 0.914 for CRC and 0.917 for AA showing a sensitivity of 79% and 75%, respectively (for 95% specificity).

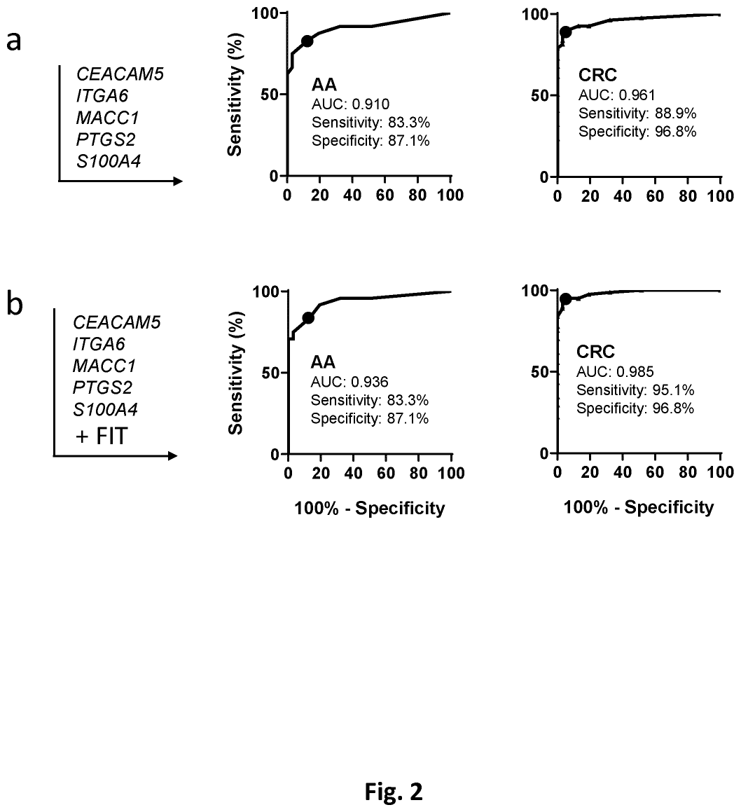

Considering that detecting 75% of the AA could be achieved using the three markers of the second group (i.e. CEACAM5, ITGA6 and MACC1), various combinations of markers belonging to the first group were included in order to improve CRC detection using a maximum of 5 targets (Table 2). Results showed that adding the two markers S100A4 and PTGS2 significantly improved the rate of CRC detection up to 89% (for 95% specificity) (FIG. 2A). Interestingly, considering the result of the FIT in combination with the multi-target score further increased CRC detection up to 95% (for a 97% specificity) but had no significant effect on AA detection (FIG. 2B).

TABLE 2
Selection of the best combinations of targets. Sensitivities and Specificities were determined based on optimal
cutoff values.

| | AA | | | | | CRC | | | | |
|---|---|---|---|---|---|---|---|---|---|---|
| | | | | | Sensi- | | | | | Sensi- |
| | | | | | tivity for | | | | | tivity for |
| | | Sensi- | Specifi- | Youden | specifi- | | Sensi- | Specifi- | Youden | specifi- |
| | AUC | tivity | city | Index | city >95% | AUC | tivity | city | Index | city >95% |
| GADD45B/ITGA2/MYBL2/MYC/PTGS2/S100A4 | .819 | 79.1 | 87.10 | .66 | 45.30 | .969 | 85.19 | 96.97 | .86 | 85.19 |
| CEACAM5/ITGA6/MACC1 | .917 | 91.67 | 83.87 | .76 | 75.00 | .914 | 79.01 | 96.97 | .76 | 79.01 |
| CEACAM5/ITGA6/MACC1 + GADD45B | .900 | 75.00 | 87.88 | .63 | 70.83 | .923 | 79.01 | 96.97 | .76 | 79.01 |
| CEACAM5/ITGA6/MACC1 + ITGA2 | .900 | 79.17 | 90.91 | .70 | 70.83 | .929 | 83.95 | 96.97 | .81 | 83.95 |
| CEACAM5/ITGA6/MACC1 + MYBL2 | .915 | 79.17 | 93.94 | .73 | 75.00 | .924 | 85.19 | 93.94 | .79 | 80.25 |
| CEACAM5/ITGA6/MACC1 + MYC | .918 | 83.33 | 93.94 | .77 | 66.67 | .939 | 85.19 | 94.94 | .79 | 80.25 |
| CEACAM5/ITGA6/MACC1 + PTGS2 | .905 | 79.17 | 90.32 | .70 | 66.67 | .944 | 86.42 | 93.55 | .80 | 81.48 |
| CEACAM5/ITGA6/MACC1 + S100A4 | .910 | 79.17 | 93.94 | .73 | 75.00 | .952 | 86.42 | 93.94 | .80 | 81.48 |
| CEACAM5/ITGA6/MACC1 + ITGA2/S100A4 | .897 | 79.17 | 90.91 | .69 | 70.83 | .958 | 86.42 | 93.94 | .80 | 82.72 |
| CEACAM5/ITGA6/MACC1 + ITGA2/PTGS2 | .890 | 75.00 | 93.55 | .69 | 66.67 | .952 | 87.65 | 93.55 | .81 | 83.95 |
| CEACAM5/ITGA6/MACC1 + PTGS2/S100A4 | .910 | 83.33 | 87.10 | .70 | 75.00 | .961 | 88.89 | 96.77 | .86 | 88.89 |
| S100A4/PTGS2 | .773 | 70.80 | 78.79 | .50 | 29.17 | .949 | 93.75 | 78.78 | .73 | 80.25 |

AUC: Area under the curve, AA: Advanced adenoma, CRC: Colorectal cancers stage I, II and III.

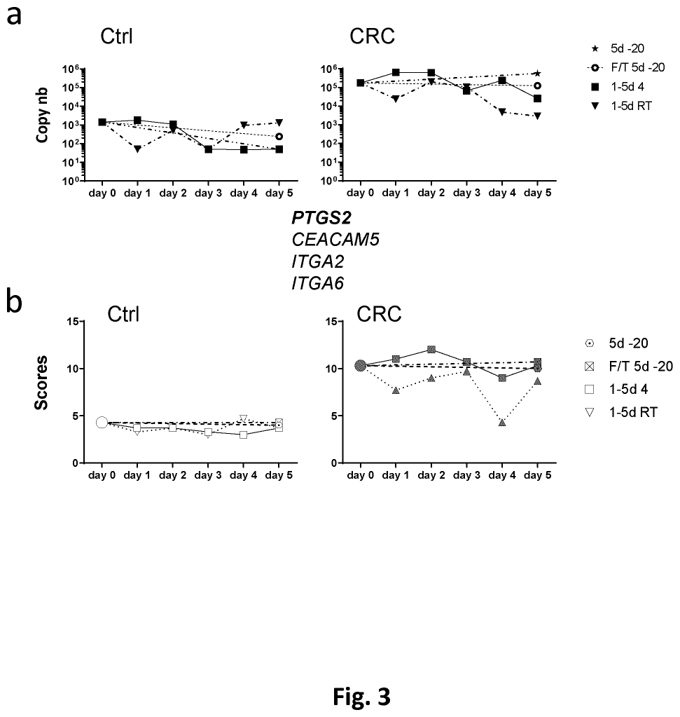

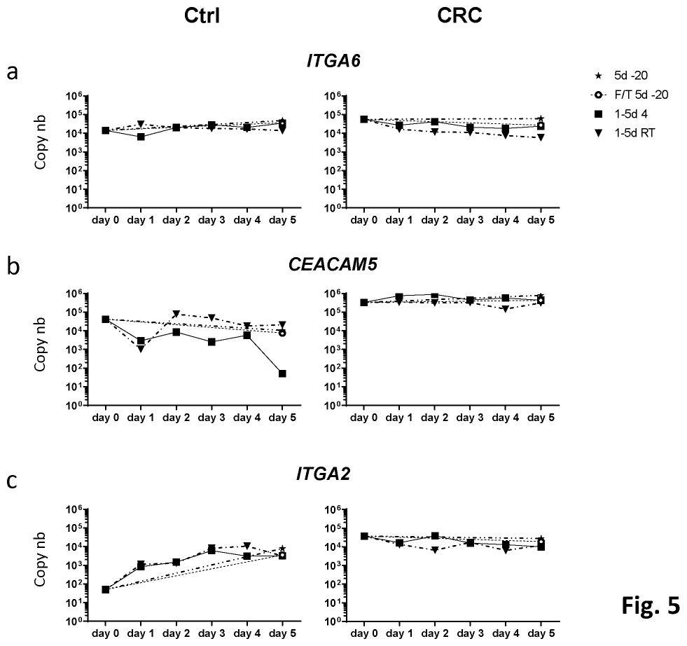

Considering that the ultimate goal would be to evaluate the feasibility of using the multi-target mRNA stool test in a clinical set-up, the stability of the mRNA targets in stool samples was evaluated subjected to various conditions of preservation that mimic the clinical reality. Stool samples were obtained from three controls and three patients diagnosed with CRC. Four of the identified targets in stools were selected for testing, including two for each group identified above: CEACAM5, ITGA6, ITGA2 and PTGS2. Conditions to be tested included conventional freezing at −20° C. with and without a thaw cycle, conservation at 4° C. and conservation at room temperature (23° C.), for a 5-day period. As shown for PTGS2 (FIG. 3A) as well as CEACAM5, ITGA6 and ITGA2 (FIG. 5), the mRNA targets were found to be very stable under all frozen and cooled conditions over the 5-day period while some variations were observed at room temperature for some markers such as PTGS2 (FIG. 3A). Score compilation of the data confirmed the relative stability of the targets for all conditions including ambient temperature for at least 3 days (FIG. 3B).

In this example, a multitarget stool mRNA test was shown to represent a powerful assay for detecting patients with colorectal cancers and demonstrate its usefulness to also detect high risk adenomas. One interest of the procedure relies on its relative simplicity considering that high sensitivities and specificities can be obtained with a selection of only five targets, thus compatible with multiplex PCR in stool samples, an approach already in place in the clinic to investigate gastrointestinal infections.

One strength of the multitarget stool mRNA test presented herein is that transcripts are directly isolated from the stools by conventional extraction methods thus being compatible with automation rather than procedures that require enrichment protocols for exfoliated colorectal cells prior to RNA extraction and processing. Another strength is the relatively low number of targets required to optimize the assay. It is worth mentioning that an important part of this proof-of-concept study was finding specific targets to identify samples from patients with AA among others that appear to be overrepresented in CRC and then selecting the strongest combination to allow the detection of both AA and CRC.

It is interesting to contextualize the findings that this study, relying on the use of only five mRNA targets, allowed the detection of 75% of the samples obtained from patients with AA and 89% of the samples obtained from patients with CRC, using a specificity of >95%. It was chosen to express the data using this optimal specificity which generates less than 5% of false positives in order to allow a fair comparison to other tests. Incidentally, integration of the FIT component to the mRNA data increased CRC sensitivity up to 95%, consistent with the fact that the origins of exfoliated cells and blood in the stools are likely to be different. Overall, a multi-target stool mRNA-FIT test allows the detection of 75% of the AA and 95% of the CRC with less than 4% of false positives. These numbers compared advantageously to any other screening test for colorectal cancerous lesions. As shown with the inclusion of the FIT component, diversification of target types improves sensitivity.

Another finding is the possibility to include a factor for predicting AA vs CRC, which could provide pertinent information ahead of colonoscopy. Indeed, considered separately, the combination of the three targets CEACAM5, ITGA6 and MACC1 selected to predict AA provided 75% and 79% sensitivity (for 95% specificity) for AA and CRC respectively and the two targets S100A4 and PTGS2 selected to improve CRC detection provided 29% and 80% sensitivity (for 95% specificity) for AA and CRC prediction, suggesting that using distinct repertoires of targets for AA and CRC could be used to improve patient stratification for colonoscopy. Specific analysis of S100A4 and PTGS2 scores for patients identified as positive in the multi-target stool mRNA test could contribute to discriminating between patients carrying AA vs those with CRC considering that, for instance, a patient with a score of >4.5 for S100A4 and PTGS2 displays a 17% probability of having a AA vs 73% odds of having a CRC.

Finally, the assessment of target stability revealed that stool sample collection to perform the multitarget stool mRNA test does not require particular conditions, being relatively stable for at least 3 days, even at room temperature. Part of this relatively surprising observation may result from the possibility that mRNA degradation is prevented in exfoliated cells, which are the main source of host mRNA in the stools. Another part results from the procedure used for selecting the mRNA targets. Incidentally, it was not surprising that only half of the 27 selected targets were amplified in stool samples. The efficient amplification of these targets was also dependent on the use of the TaqMan Gene Expression Assay which was found to be more sensitive and specific than conventional qPCR for stool samples while requiring relatively short intact mRNA sequences.

In conclusion, this example demonstrates the usefulness of host mRNAs as biomarkers to identify patients carrying curable colorectal cancers as well as precancerous lesions.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Dydensborg A B, Herring E, Auclair J, Tremblay E, Beaulieu J-F. Normalizing genes for quantitative RT-PCR in differentiating human intestinal epithelial cells and adenocarcinomas of the colon. Am J Physiol Gastrointest Liver Physiol 2006; 290:G1067-1074.
Beaulieu J F, Herring E, Kanaoka S, Tremblay E. Use of integrin alpha 6 transcripts in a stool mRNA assay for the detection of colorectal cancers at curable stages. Oncotarget 2016; 7:14684-92.
Hamaya Y, Yoshida K, Takai T, Ikuma M, Hishida A, Kanaoka S. Factors that contribute to faecal cyclooxygenase-2 mRNA expression in subjects with colorectal cancer. Br J Cancer 2010; 102:916-21.
Herring E, Kanaoka S, Tremblay E, Beaulieu J F. A stool multitarget mRNA assay for the detection of colorectal neoplasms. Methods Mol Biol 2018; 1765:217-227.
Herring E, Kanaoka S, Tremblay E, Beaulieu J F. Droplet digital PCR for quantification of ITGA6 in a stool mRNA assay for the detection of colorectal cancers. World J Gastroenterol 2017; 23:1-8.

What is claimed is:

1. A method of stratifying the risk of a subject of having an advanced colorectal adenoma or a colorectal cancer in a subject, the method comprises:
a) providing a stool sample from the subject, wherein the stool sample comprises a plurality of mRNA transcripts from the subject;
b) determining the mRNA expression level of at least two distinct genes from the plurality of mRNA transcripts to obtain a test expression profile; and
c) comparing the test expression profile with a control expression profile, wherein the control expression profile comprises the mRNA expression level of the at least two genes and is derived from a plurality of control mRNA transcripts from a control subject known to lack the advanced colorectal adenoma or the colorectal cancer;
wherein if it is determined that the test expression profile of the subject comprises at least two genes whose expression are increased with the respect to the control expression profile, the subject is stratified as having an increased risk of having the advanced colorectal adenoma or the colorectal cancer, when compared to the control subject.

2. The method of claim 1, wherein the stool sample comprises at least one colorectal epithelial cell.

3. The method of claim 2, wherein the at least one colorectal epithelial cell comprises the plurality of mRNA transcripts.

4. The method of any one of claims 1 to 3, wherein the test expression profile and the control expression profile comprise the mRNA expression level of at least two of the S100A4 gene, the GADD45B gene, the ITGA2 gene, the MYBL2 gene, the MYC gene, the CEACAM5 gene, and/or the MACC1 gene.

5. The method of any one of claims 1 to 4, wherein step b) comprises determining the mRNA expression level from at least one additional gene from the plurality of mRNA transcripts, wherein the test expression profile and the control expression profile further comprise the expression level of the PTGS2 gene and/or of the ITGA6 gene.

6. The method of any one of claims 1 to 5, wherein the test expression profile and the control expression profile comprise the mRNA expression level of the CEACAM5 gene, the ITGA6 gene and/or the MACC1 gene.

7. The method of any one of claims 1 to 5, wherein the test expression profile and the control expression profile comprise the mRNA expression level of the S100A4 gene, the GADD45B gene, the ITGA2 gene, the MYBL2 gene, the MYC gene and/or the PTGS2 gene.

8. The method of any one of claims 1 to 7, wherein step b) comprises using a reverse-transcriptase polymerase chain reaction (RT-PCR) to obtain the mRNA expression level of the at least two genes of the test expression profile and/or the control expression profile.

9. The method of any one of claims 1 to 8, wherein step b) comprises using a quantitative polymerase chain reaction (qPCR) to obtain the mRNA expression level of the at least two genes of the test expression profile and/or the control expression profile.

10. The method of any one of claims 1 to 9, further comprising, prior to step b), storing the stool sample.

11. The method of any one of claims 1 to 10, further comprising determining the presence of hemoglobin in the stool sample.

12. The method of claim 11 comprising using a fecal immunochemical test (FIT) to determine the presence of hemoglobin in the stool sample.

13. The method of any one of claims 1 to 12, further comprising determining the presence of a DNA mutation and/or an aberrant DNA methylation pattern associated with a predisposition to a colorectal cancer in the colorectal epithelial cell of the subject.

14. The method of claim 13, wherein the at least one DNA mutation is located in the K-RAS gene.

15. The method of claim 13 or 14, wherein the aberrant DNA methylation pattern is located in the NDRG4 gene and/or the BMP3 gene.

16. The method of any one of claims 11 to 15 comprising using the Cologuard™ assay to determine the presence of hemoglobin in the stool sample, the presence of DNA mutation and/or the presence of the abnormal DNA methylation pattern.

17. The method of any one of claims 1 to 16 for screening for subjects suitable for colonoscopy.

18. The method of any one of claims 1 to 17, further comprising submitting the subject having been stratified as being at increased risk of developing the colorectal cancer to a chemotherapy, a radiotherapy and/or a surgery.

19. The method of any one of claims 1 to 18, wherein the colorectal cancer is a colon cancer.

20. The method of any one of claims 1 to 18, wherein the colorectal cancer is a rectal cancer.

21. A kit for stratifying the risk of a subject of having an advanced colorectal adenoma or a colorectal cancer in a subject, wherein the kit comprises at least two reagents for determining the mRNA expression level of at least two distinct genes from the plurality of mRNA transcripts to obtain a test expression profile in a stool sample from the subject.

22. The kit of claim 21, further comprising a container for storing a stool sample.

23. The kit of claim 21 or 22, wherein the at least two reagents are for determining the mRNA expression level of the S100A4 gene, the GADD45B gene, the ITGA2 gene, the MYBL2 gene, the MYC gene, the CEACAM5 gene, and/or the MACC1 gene.

24. The kit of claim 23, further comprising at least one additional reagent for determining the mRNA expression level of the PTGS2 gene and/or of the ITGA6 gene.

25. The kit of any one of claims 21 to 24, wherein the at least two reagents are for determining the mRNA expression level of the CEACAM5 gene, the ITGA6 gene and/or the MACC1 gene.

26. The kit of any one of claims 21 to 24, wherein the at least two reagents are for determining the mRNA expression level of the S100A4 gene, the GADD45B gene, the ITGA2 gene, the MYBL2 gene, the MYC gene and/or the PTGS2 gene.

27. The kit of any one of claims 21 to 26, further comprising a reverse-transcriptase.

28. The kit of any one of claims 21 to 27, further means for determining the presence of hemoglobin in the stool sample.

29. The kit of claim 28, further comprising using a fecal immunochemical test (FIT) to determine the presence of hemoglobin in the stool sample.

30. The kit of any one of claims 21 to 29, further comprising reagents for determining the presence of a DNA mutation and/or an aberrant DNA methylation pattern associated with a predisposition to a colorectal cancer in the colorectal epithelial cell of the subject.

31. The kit of claim 30, wherein the at least one DNA mutation is located in the K-RAS gene.

32. The kit of claim 30 or 31, wherein the aberrant DNA methylation pattern is located in the NDRG4 gene and/or the BMP3 gene.

33. The kit of any one of claims 30 to 32, further comprising a Cologuard™ and/or a Coloalert™ assay to determine the presence of hemoglobin in the stool sample, the presence of DNA mutation and/or the presence of the abnormal DNA methylation pattern.
